(19) United States
(12) Patent Application Publication
(10) Pub. No.: US 2002/0183860 A1
(43) Pub. Date: Dec. 5, 2002

(54) Tubular prosthetic foot with elastomeric cushion

(76) Inventors: Kerry E. Wilkinson, Chandler, AZ (US); Randall S. Whiteside, Phoenix, AZ (US)

Correspondence Address: H. GORDON SHIELDS, 7830 NORTH 23RD AVENUE, PHOENIX, AZ, 85021, US

(21) Appl. No.: 09/866,294

(22) Filed: May 29, 2001

(51) Int. Cl. A61F 2/66

(52) U.S. Cl. 623/52; 623/55

(57) ABSTRACT

Prosthetic foot apparatus includes a flexible foot plate, an elastomeric block, and a tubular pylon element. The tubular pylon element includes an upper tubular portion, a partially flattened curved portion, and a generally flattened lower portion secured to the foot plate by an elastomeric block. The elastomeric block is configured to allow, with the pylon and the foot plate, inversion, eversion, dorsi flexion, and planar flexion movements of the apparatus to simulate ankle and toe movements of a normal foot. The foot plate and the tublar pylon allow for dorsi flexion and planar flexion movements, and the elastomeric block is appropriately configured to add inversion and eversion movements as well as dorsi flexion and planar flexion movements of the apparatus.

### BACKGROUND OF THE INVENTION

1. Field of the Invention

This invention relates to prosthetic apparatus and, more particularly, to a prosthetic foot and ankle apparatus utilizing an elastomeric element.

2. Description of the Prior Art

U.S. Pat. No. 6,053,946, dated Apr. 25, 2000, the inventor of which is co-inventor herein, discloses the use of a tubular element for a prosthetic foot. The apparatus allows for dorsi flexion and planar flexion movements, but does include provisions for inversion and eversion movements. A series of patents by Phillips, including U.S. Pat. No. 5,181,932, U.S. Pat. No. 5,486,209, U.S. Pat. No. 5,514,186, U.S. Pat. No. 5,593,457, and U.S. Pat. No. 5,976,191, all of which are related to each other, disclose the use of flexible foot and ankle portions, with the ankle portions demountably connected to the foot portions, also provide dorsi flexion movement and planar flexion movement. However, none of the patents include elements which provide for inversion and eversion movements. It will be noted that all of the Phillips patents also include connector elements for connecting the foot portion to pylon leg elements.

The apparatus of the present invention differs from the above referred to prior art in that it combines the advantages of the tubular elements of the '946 patent with elastomeric elements which allow for inversion and eversion movements.

### SUMMARY OF THE INVENTION

The invention described and claimed herein comprises prosthetic foot apparatus including a tubular element secured to a flexible foot plate and an elastomeric element disposed between the tubular element and the foot plate. The elastomeric element is appropriately configured to provide both inversion and eversion ankle movements and, with a tubular and foot element, also for dorsi flexion and planar flexion movements.

Among the objects of the present invention are the following:

To provide new and useful prosthetic apparatus

To provide new and useful prosthetic foot and ankle apparatus;

To provide new and useful prosthetic apparatus including a tubular member secured to a foot plate through an elastomeric element;

To provide new and useful prosthetic apparatus utilizing an elastomeric element disposed between a flexible foot plate and a tubular member; and

To provide new and useful prosthetic apparatus having a shaped elastomeric element secured to a foot plate and to a tubular member.

### BRIEF DESCRIPTION OF THE DRAWING

FIG. 1 is a perspective view of the apparatus of the present invention.

FIG. 2 is an exploded perspective view of the apparatus of FIG. 1.

FIG. 3 is a perspective view of an alternate embodiment of the apparatus of FIGS. 1 and 2.

FIG. 4 is an enlarged perspective view, partially broken away, of the apparatus of FIG. 3.

### DESCRIPTION OF THE PREFERRED EMBODIMENT

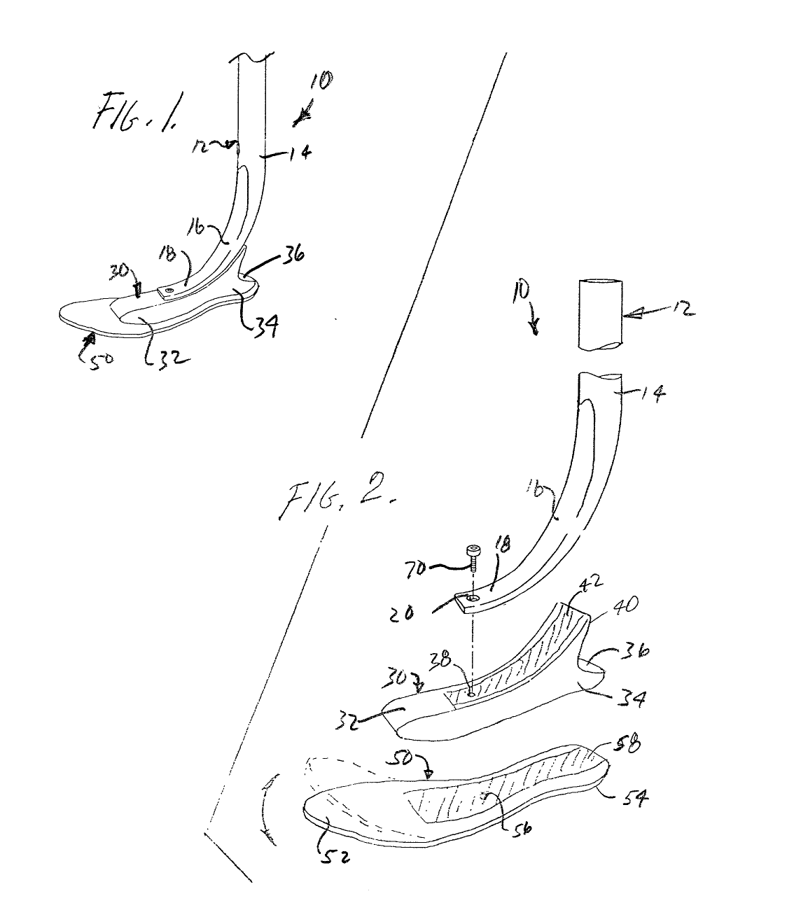

FIG. 1 is a perspective view of prosthetic foot apparatus 10 of the present invention. FIG. 2 is an exploded perspective view of the apparatus 10 of FIG. 1, showing the various elements included in the prosthetic apparatus 10. For the following discussion, reference will be made to both FIGS. 1 and 2.

Prosthetic foot apparatus 10 includes a tubular pylon element 12 in which an upper portion 14 is of a convention tubular configuration, but the tubular configuration 14 includes a curved portion 16 in which the tubular portion 14 starts to be flattened. The flattening is completed at a lower foot portion 18.

The flattened lower foot portion 18 is secured by an appropriate fastener, such as a screw 70, through an elastomeric block element 30 to a foot plate 50. The screw 70 extends through a hole or aperture 20 in the flattened lower foot portion 18.

The elastomeric block 30 includes a front toe portion 32 and a rear heel portion 34. The elastomeric block 30 extends upwardly from the toe portion 32 to an upper heel portion 40. Between the upper heel portion 40 and the lower heel portion 34 is a relieved area 36. The purpose of the relieved area 36 is to provide a desired flexibility in the elastomeric block 30. Shown in FIG. 2 is a shaded area 42 on the elastomeric block 30 which corresponds to the portion of the elastomeric block 30 which is in contact with the curved portion 16 and the flat portion 18 of the pylon 12. Adjacent to the front or lower portion of the shaded area 42 is a hole or aperture 38 into which the screw 70 extends.

The foot plate 50 may be metal or may be any other appropriate material that allows for dorsi flexion and planar flexion movements. The foot plate 50 includes a front portion 52, and a heel portion 54. A screw hole 56 may receive the bottom portion of the screw 70 to secure the toe portion 18 of the pylon 12, the elastomeric block 30, and the foot plate 50 together.

Also illustrated in FIG. 2 is a shaded portion 58 which corresponds to the portion of the foot plate 50 which receives the bottom of the elastomeric block 30.

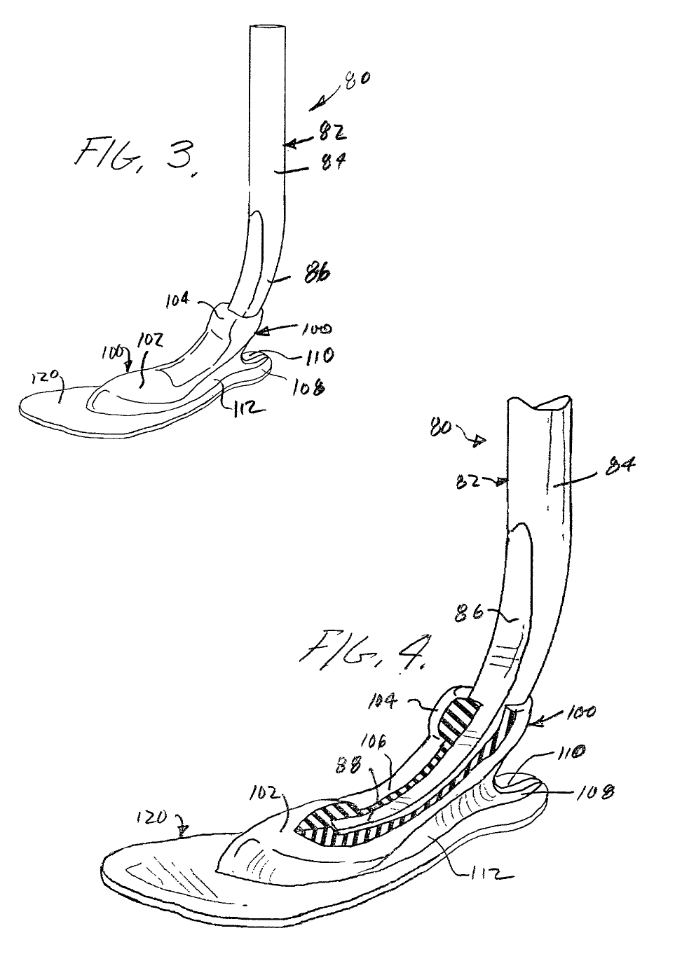

FIG. 3 is a perspective view of an alternate embodiment of the apparatus of FIGS. 1 and 2. FIG. 4 is an enlarged perspective view of the apparatus of FIG. 3, with a portion partially broken away. For the following discussion, reference will be made to both FIGS. 3 and 4.

FIGS. 3 and 4 disclose prosthetic foot apparatus 80, which comprises an alternative embodiment of the apparatus 10 of FIGS. 1 and 2. Apparatus 80 includes a tubular pylon 82 which essentially includes three portions, an upper tubular portion 84, a curved, partially flattened tubular portion 86, and a generally flattened foot portion 88. The generally tubular pylon 82 is secured to a flexible foot plate 20 through an elastomeric block 100.

The tubular pylon 82 is substantially identical, generally to the pylon 12 of the apparatus 10. The foot plate 120 is substantially identical to the foot plate 50. However, the elastomeric block 100 is different from the elastomeric block 30 of FIGS. 1 and 2. The difference is primarily in the configuration of the elastomeric block 100. As noted in both FIGS. 3 and 4, as contrasted with FIGS. 1 and 2, part of the pylon 82 is disposed within the elastomeric block 100, as opposed to being disposed on top of the elastomeric block 30 for the pylon 12 of FIGS. 1 and 2.

A configuration of the elastomeric block 100 is accordingly different from the configuration of the elastomeric block 30 of FIGS. 1 and 2.

The elastomeric block 100 includes a toe portion 102, an ankle portion 104, an upper foot portion 106, and a heel portion 108. The ankle portion 104 is an enlarged, bulbous portion which helps to anchor the pylon 82 to the block 100. At the same time, the ankle portion 104 allows for movement to help simulate an ankle movement of a normal foot.

The upper foot portion 106 is relatively thin so as to allow for dorsi flexion an planar flexion movements of the apparatus 80. The toe portion 102 is thickened to limit dorsi flexion and planar flexion movement and also to help anchor the pylon 82 to the foot plate 102.

There are two primary relieved areas in the elastomeric block 100. These include a relieved heel portion 110 and a relieved side area 112. The relieved portion 110 helps to allow the dorsi flexion and planar flexion, and the relief portion 112 helps to control inversion and eversion movements of the apparatus 80.

Obviously, the extent of the inversion, eversion, dorsi flexion, and planar flexion movements of the apparatus 80 may be varied to suit a particular user and to suit the desired use of manner of using of the apparatus 80 by a particular user. Thus, for walking, there may be one particular configuration, while for running or participating in other sporting type activities, the configuration of the elastomeric block 100 may vary. Moreover, the flexibility of the foot plate 120 may also vary, along with the configuration of the elastomeric block 100, according to the desires of a user. Obviously, the same may be said for the configuration or size of the pylon 82, with its upper tubular portion, its curve, partially flattened portion 86, and its flattened foot portion 88. And, obviously, the same may be said for the pylon 12 of FIGS. 1 and 2, described above.

The configuration of the foot plates 50 and 120 may also vary, in particularly the thickness, to accommodate users of various sizes and different purposes to which the prosthetic apparatus may be put by a particular user.

While the principles of the invention have been made clear in illustrative embodiments, there will be immediately obvious to those skilled in the art many modifications of structure, arrangement, proportions, the elements, materials, and components used in the practice of the invention, and otherwise, which are particularly adapted to specific environments and operative requirements without departing from those principles. The appended claims are intended to cover and embrace any and all such modifications, within the limits only of the true spirit and scope of the invention.

What I claim is:

1. Prosthetic foot apparatus comprising in combination:
a flexible foot plate for providing dorsi flexion and planar flexion movements; pylon means, including
a first tubular portion,
a second partially flattened curved portion extending from the first tubular portion, and
a third flattened portion extending from the second partially flattened portion; and
elastomeric block means secured to both the flexible foot plate and the pylon means for providing eversion and inversion movements.

2. The apparatus of claim 1 in which the elastomeric block means is disposed between the third flattened portion of the pylon means and the flexible foot plate.

3. The apparatus of claim 1 in which the elastomeric block means includes a toe portion and a heel portion.

4. The apparatus of claim 3 in which the elastomeric block means further includes a relieved portion at the heel portion.

5. The apparatus of claim 1 in which the third flattened portion of the pylon means is disposed in the elastomeric block means.

6. The apparatus of claim 5 in which the elastomeric block means includes a plurality of relieved portions to provide desired eversion and inversion movements.
